(19) United States
(12) Patent Application Publication
(10) Pub. No.: US 2008/0216827 A1
(43) Pub. Date: Sep. 11, 2008

(54) INTUBATION DEVICES AND METHODS OF USE THEREOF

(76) Inventors: Anna S. Seydel, Bonita, CA (US); Donald J. Chalmers, Chula Vista, CA (US)

Correspondence Address: LAW OFFICE OF MICHAEL P. EDDY;MICHAEL P. EDDY, 12526 HIGH BLUFF DRIVE, STE. 300, SAN DIEGO, CA, 92130, US

(21) Appl. No.: 12/045,679

(22) Filed: Mar. 10, 2008

Related U.S. Application Data
(60) Provisional application No. 60/905,987, filed on Mar. 9, 2007.

(51) Int. Cl. A61M 16/00

(52) U.S. Cl. 128/200.26; 128/202.22

(57) ABSTRACT

An intubation device having a longitudinal, flexible frame with a leader guide positioned at the distal region of the frame and at least one restraint attached to the frame that is adapted to hold and guide a medical tube. The device facilitates the process of inserting a medical tube into a body cavity so as to achieve minimal patient discomfort. The intubation device described herein allows a medical professional to insert a nasogastric tube into a patient's nose, past the soft palate, and into the esophagus while significantly lessening the chances for error and alleviating the problems of tissue abrasion and improper insertion.

### CROSS-REFERENCES TO RELATED APPLICATIONS

This application claims the benefit of U.S. provisional application Ser. No. 60/905,987, filed Mar. 9, 2007 which is incorporated herein by reference in its entirety for all purposes.

### STATEMENT REGARDING FEDERALLY SPONSORED RESEARCH OR DEVELOPMENT

None.

### REFERENCE TO SEQUENCE LISTING, A TABLE, OR A COMPUTER PROGRAM LISTING COMPACT DISK APPENDIX

None.

### BACKGROUND

1. Field

The claimed subject matter relates to intubation devices that facilitate insertion into and use of the devices in conjunction with patients, as well as combinations of the device and such a tube and methods of using the device and the combinations.

2. Description of the Related Art

Nasogastric (NG) tubes are used in patient care for introducing and/or evacuating gases, liquids and solids to and from the gastrointestinal tract, particularly the stomach and upper small intestine. Such devices are commonly employed, for example, at the postoperative stage of abdominal surgery to empty the stomach of secretions and gas in order to prevent gastric dilation. They are also used for attaining adequate nutrition for patients unable to take oral nourishment. Nasogastric intubation and feeding may be prescribed, for example, when the normal digestive mechanism is impaired. Impairment may have many causes, ranging from localized digestive tract trauma to loss of autonomic function.

Insertion into and use of nasogastric tubes in conjunction with patients pose various discrete problems. Nasogastric tubes are often difficult to introduce because of patient anatomy. Typically, nasogastric tubes are inserted by force through the nasal passageways, causing the patient considerable discomfort due to tearing and abrasion of the tissues in, for example, the nostrils, nasopharynx, hypo-pharynx, and esophagus. Despite their frequent use, nasogastric tubes are associated with numerous complications, for example, lung aspiration, pneumothorax, and coiling.

Improper insertion of a nasogastric tube may lead to physical damage and trauma at a number of locations. For example, one area of concern is the region of the ethmoid bone, which separates the nasal cavity from the brain. A nasogastric tube inserted into the nose is capable of penetrating through the ethmoid bone and into the brain, resulting in possible brain damage to the patient, or even death.

A further area of concern upon inserting a nasogastric tube is fitting the tube into the esophagus to lead it into the stomach. If the tube is inadvertently inserted past the epiglottis and into the trachea instead of the esophagus, damage to the larynx, vocal cords, and/or lungs may result. Nasogastric tubes may have a curved shape in order to guide them past the nasopharynx. However, this curved shape has the tendency to lead the nasogastric tube into the trachea rather than the esophagus.

Additional problems may arise as the terminal end of the tube is introduced into the stomach. The tube may form a loose knot upon itself while it is located in the stomach. This knot may tighten when the tube is removed. A knot in the distal end of the nasogastric tube may lead to extreme patient discomfort when the tube is to be removed, and may lead to increased tearing and abrasion in various tissues.

Attempts to remedy the problems of nasogastric tubes have been insufficient to address one or more of these discrete problems. U.S. Pat. No. 5,690,620 to Knott discloses an NG tube with a curved or bent leading end. The leading end of the NG tube of Knott is biased to conform to the shape of the soft palate and allows for passage into the esophagus of the patient, but only if the NG tube is rotated 180 degrees at a particular point during insertion. U.S. Pat. No. 5,700,252 to Klingenstein discloses an NG tube with a tapered tip having less strength than the remaining portion of the tube. However, such tip construction is difficult and also interferes with suction and feeding performance. Thus, the art is in need of alternative methods and equipment for inserting and using a nasogastric tube.

### SUMMARY

Intubation devices are described that facilitate the process of inserting a nasogastric tube so as to achieve minimal patient discomfort. One embodiment of an intubation device described herein allows for insertion of a nasogastric tube into a patient's nasal pathway, past the soft palate, and into the esophagus while significantly reducing the chances for error and alleviating one or more of the problems described above.

In an embodiment, the intubation device comprises a longitudinal, flexible frame comprising a distal region and a proximal region with a leader guide positioned at the distal region of the frame, wherein said leader guide is in the form of a soft pliable curvature, and at least one restraint attached to the frame that is adapted to hold a medical tube.

In another embodiment, a nasogastric tube assembly is described comprising an intubation device, preferably as described in the preceding paragraph, together with a medical tube comprising a distal region and a proximal region. In some embodiments, the medical tube comprises a nasogastric tube.

A medical tube is also described for use alone or in the combination of the previous paragraph, wherein the medical tube is adapted to be used with an intubation device as described herein. In another embodiment, the present invention provides a medical kit comprising packaging material, an intubation device as described herein, and a medical tube.

A method of inserting a medical tube is also described comprising providing an intubation device comprising a longitudinal, flexible frame comprising a distal region and a proximal region; a leader guide positioned in the distal region of the frame, wherein said leader guide is in the form of a soft pliable curvature; and at least one restraint attached to the frame and adapted to hold a medical tube; providing a medical tube comprising a distal region and a proximal region; fitting the tube into the intubation device; inserting the intubation device and the tube into a nasal cavity such that the terminal end of the distal region of the tube is located between the soft palate region and the opening of the esophagus; and withdrawing the intubation device while holding the tube in place.

### BRIEF DESCRIPTION OF THE DRAWINGS

FIG. 1 shows a top view of an intubation device according to an embodiment of the claimed subject matter.

FIGS. 1A through 1C are cross-sections of an embodiment of the intubation device shown in FIG. 1.

FIG. 1D is a cross-section of an embodiment of the intubation device according to FIG. 1.

FIG. 2 shows a side view of an intubation device according to an embodiment of the claimed subject matter.

FIG. 3 shows a cross-section of the head and neck of a human patient undergoing administration of a nasogastric tube using an intubation device as described herein.

FIG. 4 illustrates the components of another embodiment of the claimed subject matter.

### DETAILED DESCRIPTION OF THE EMBODIMENTS

In an embodiment, the intubation device comprises a longitudinal, flexible frame comprising a distal region and a proximal region. A leader guide is positioned on the distal region of the frame, and the leader guide is preferably in the form of a soft pliable curvature. At least one restraint is preferably attached to the frame and adapted to hold a medical tube, such as a nasogastric tube.

Described herein is an intubation device comprising a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube.

In one embodiment, the intubation device comprises a plastic or rubber material and comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube, wherein the intubation device comprises a plastic or rubber material. The plastic material is preferably selected from the group consisting of polyvinyl chloride, silicone, polyurethane, polyethylene, polypropylene, polycarbonate, polyester, polyacrylate, polytetrafluroroethylene, and combinations and copolymers thereof.

In another embodiment, the intubation device comprises a longitudinal, flexible frame that maintains a substantial lineal integrity, and further comprises a distal region and a proximal region, a leader guide positioned at the distal region of the frame, wherein said leader guide is in the form of a soft pliable curvature; and at least one restraint attached to the frame and adapted to hold a medical tube. The frame is, preferably, capable of a 180 degree direction reverse within no more than about a 4 cm radius. Also preferably, the frame is capable of a 180 degree direction reverse within no more than about a 3, 2, or 1 cm radius. Alternatively, the leader guide retains a curvature memory. Further alternatively, the curvature of the leader guide is shaped to permit the leader guide to substantially follow the natural arch of a body cavity, for example, an upper nasal cavity.

In another embedment, the intubation device comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube, wherein at least one of the tube restraints comprises a substantially circular or substantially semi-circular shape. Alternatively, at least one of the tube restraints is independently selected from the group comprising a substantially closed guide configuration restraint, a substantially semi-closed guide configuration restraint, and a substantially open guide configuration restraint.

Alternatively, in another embodiment, the intubation device comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube, wherein at least one distal tube restraint is positioned in the distal region of the frame and at least one proximal tube restraint is positioned in the proximal region of the frame. The at least one distal tube restraint and the at least one proximal tube restraint may, preferably, be adapted to maintain substantially lateral alignment of a medical tube with the intubation device. Preferably, the at least one distal tube restraint comprises a substantially open guide or a substantially semi-closed guide. The intubation device preferably comprises one, two or three distal tube restraints, and preferably comprises one, two or three proximal tube restraints. The first proximal tube restraint is preferably closer to a terminal end of the distal region of the intubation device than any other proximal tube restraint. Preferably, the distance between the terminal end of the distal region and the first proximal tube restraint is adapted so that when the intubation device is inserted into a nasal cavity, the terminal end of the distal region will be positioned between the soft palate region and the opening of the esophagus when the first proximal restraint is positioned at the opening of the nostril.

In another embodiment, the intubation device comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube, and further comprises a lubricant.

Embodiments of the intubation device may be made of any material that is useful and safe for administration into a human or other mammalian body. In one embodiment, the intubation device comprises a plastic or rubber material. Preferably, in these embodiments, the intubation device is suitable for sterilization and, optionally, repeated sterilization. When used, the intubation device is preferably sterilized or manufactured with sterile material. Further, the material is stable in the nasal, oral, and/or digestive environment for extended periods of time. Non-limiting examples of materials useful in forming the intubation include various medically-suitable polymers, such as polyvinyl chloride, silicone, polyurethane, polyethylene, polypropylene, polycarbonate, polyester, polyacrylate, polytetrafluroroethylene, and combinations and copolymers thereof. Other embodiments include disposable devices.

In an embodiment, the frame of the intubation device maintains a substantial lineal integrity. By “substantial lineal integrity” it is meant that the frame remains substantially straight in its configuration without the application of pressure to the device. The frame, however, is sufficiently flexible so that when pressure is applied at one end, typically the distal end, the frame will bend to accommodate the pressure. For example, when the intubation device is inserted into a body cavity, pressure may be applied to the distal end in the form of resistance from the body cavity. The frame of the intubation device is flexible enough to bend so that the distal end does not penetrate the body cavity. At the same time, the frame possesses the lineal integrity to guide itself along the body cavity that is applying resistance to it.

The flexibility of the frame of the embodiments of the intubation device may be varied depending upon the size and type of medical tube to be inserted into the patient's body and the dimensions of the cavity where the tube is being inserted. For medical tubes that are relatively less stiff and/or have a smaller diameter, the frame may comprise a material of commensurate higher flexibility. For medical tube embodiments that are relatively stiff and/or have larger dimensions, a frame with commensurate less flexibility or increased hardness may be required. Generally, greater amounts of flexibility are desired because the more flexible or soft the intubation device is, the less damage it is likely to cause to the bones and tissues of the body cavity where the medical tube is to be inserted.

In one embodiment, the frame is capable of a 180 degree direction reverse within no more than about a 4 cm radius. In another embodiment, the frame is capable of a 180 degree direction reverse within no more than about a 3 cm radius. In another embodiment, the frame is capable of a 180 degree direction reverse within no more than about a 2 cm radius. In another embodiment, the frame is capable of a 180 degree direction reverse within no more than about a 1 cm radius. The flexibility of the frame may be adjusted by altering the material used to form the intubation device by, for example, selecting the appropriate polymer and/or by altering the thickness dimension of the frame. Increased thickness of the intubation device results in increased strength and decreased flexibility.

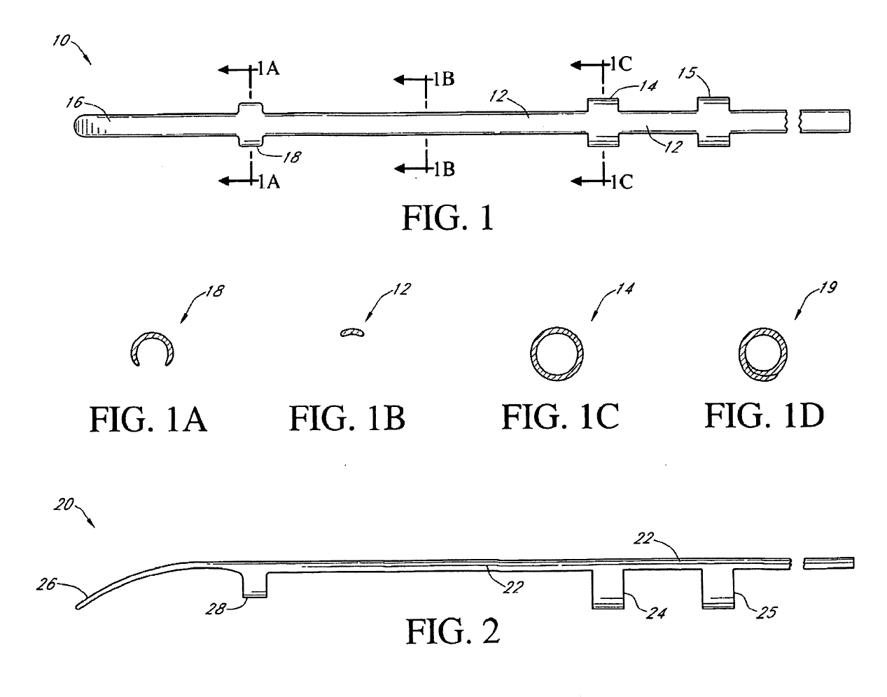

FIG. 1 shows the top view of an intubation device according to a non-limiting embodiment of the present invention. The intubation device (10) comprises a frame (12) comprising a distal region and a proximal region. A leader guide (16) is positioned at the distal region of the frame (12) and is in the form of a soft pliable curvature. The intubation device also comprises at least one, more preferably two, and in the embodiment depicted in FIG. 1, three restraints (14, 15, 18) adapted to hold a medical tube. Additional restraints may be employed.

In an embodiment, the intubation device comprises at least one distal tube restraint (18) positioned in the distal region of the frame and at least one proximal tube restraint (14, 15) positioned in the proximal region of the frame. The at least one distal tube restraint works together with the at least one proximal tube restraint to hold a medical tube in substantial lateral alignment with the intubation device. When a medical tube is removably attached to the intubation device (10), the medical tube can be aligned by a medical practitioner or assistant with the frame (12) of the intubation device (10). The tube is most preferably held in place via the proximal tube restraints (14, 15) and the distal tube restraint (18).

One or more than one proximal tube restraint may be used to maintain alignment of the medical tube during insertion into a patient. Further, the restraints allow the medical tube to be removably attached to the intubation device. For example, FIG. 1 shows one embodiment where two proximal tube restraints (14, 15) are used. In an embodiment, the intubation device comprises between one and three proximal tube restraints, although many more restraints are contemplated. For example, where the tube restraints are designed with smaller width dimensions and less surface area for holding onto a medical tube, then up to four, five, six, seven, eight, nine, or even ten proximal restraints may be used. The possible number of proximal tube restraints should not be limited, but should be sufficient to hold the removably attached medical tube in alignment during intubation into a patient.

Additionally, more than one distal tube restraint (18) may be used to maintain a better hold on the medical tube during insertion into a patient. In an embodiment, the intubation device comprises between one and three distal tube restraints, although many more restraints are contemplated. For example, where tube restraints are designed with smaller width dimensions and less surface area for holding onto a medical tube, then up to four, five, six, seven, eight, nine, or even ten proximal restraints may be used. The possible number of distal tube restraints should not be limited, but should be sufficient to hold the removably attached medical tube in alignment during intubation into a patient.

In the described embodiments, the distal tube restraints are more likely to come into contact with the internal bones and tissues of a patient upon insertion into the body. The distal tube restraints may, thus, have smaller dimensions than the proximal tube restraints in order to reduce the likelihood and severity of tissue abrasion and related damage. For example, the distal tube restraints may be formed with smaller thicknesses than the proximal tube restraints. Additionally, the distal tube restraints may be formed with smaller length and/or width than the proximal tube restraints.

The cross-sectional shape of both the proximal tube restraints and the distal tube restraints may vary so long as the restraint is capable of assisting the intubation device to hold a medical tube. Generally, but not necessarily, medical tubes and nasogastric tubes are substantially circular or elliptical in cross-sectional shape. The cross-sectional shape of the tube restraint may, thus, also be substantially circular or substantially elliptical and the medical tube fits snugly against the restraint. Other cross-sectional tube restraint shapes, such as substantially semi-circular, substantially rectangular, substantially triangular, or any other polygonal configuration are contemplated and may be selected to suit tubes of similar cross-sectional shapes. In addition, the tube restraint may take the form of at least two prongs which extend from the frame in any fashion such that the medical tube may be placed and held within the prongs. Such restraints are termed, “V-shaped.” Such prongs may also have any cross-sectional form, such as substantially circular, substantially semi-circular, substantially elliptical, linear, or substantially linear. The proximal tube restraints and the distal tube restraints may comprise similar cross-sectional shapes or the shapes may each be independently selected for each individual restraint.

The cross-sectional configuration of the proximal tube restraints and the distal tube restraints may also vary. The restraints may take on guide configurations that can be classified as one of open, closed, or semi-closed.

FIG. 1A is a cross-sectional view of the distal tube restraint (18) from FIG. 1. This tube restraint (18) may be classified as “open” because it does not form a completely closed loop of material. An open cross-sectional configuration provides a gripping action for retaining the medical tube. Also, the open bottom also allows for easier removal of the intubation device from the medical tube after the medical tube has been installed into the patient. In one embodiment, the distal tube restraint (18) comprises an open cross-sectional guide configuration. This embodiment has the advantage of easier removal when the distal end of the intubation device is deep within a body cavity. Additionally, the restraint is less likely to cause tearing and abrasion to the body tissues because of the open bottom. Although it is contemplated that one, more, or all distal tube restraints may comprise the open guide configuration, it is also contemplated that one, more, or all of the proximal tube restraints also may comprise the open guide configuration.

FIG. 1B is a cross-sectional view of the frame (12) from FIG. 1. In the embodiment displayed by FIG. 1B, the frame has a rounded cross-section. This is particularly useful when the intubation device (10) is being inserted into a body cavity that also has an elliptical or rounded shape. Preferably, the frame device will be shaped and designed to the shape of the body cavity to which it is being inserted. Cross-sectional shapes besides other than elliptical or substantially elliptical are contemplated for the frame, including, for example substantially circular, substantially rectangular, substantially triangular, or any other polygonal configuration. Preferably, the cross-sectional shape of the frame may be designed to be free of sharp edges or burrs that can abrade or damage tissue in the body. Even more preferably, all edges from the frame, tube restraints, and leader guide will be rounded to prevent tissue abrasion.

FIG. 1C is a cross-sectional view of the proximal tube restraint (14) from FIG. 1. This tube restraint (14) may be classified as “closed” because the material forms a complete loop without having any opening. A closed cross-sectional guide configuration provides improved gripping for retaining the medical tube. In one embodiment, the proximal tube restraint (14) comprises a closed cross-sectional guide configuration. This embodiment has the advantage of restraining any lateral movement of the medical tube. Although it is contemplated that one, more, or all proximal tube restraints may comprise the closed guide configuration, it is also contemplated that one, more, or all of the distal tube restraints also comprise the closed guide configuration.

FIG. 1D is a cross-sectional view of an alternative proximal or distal tube restraint (19). This tube restraint (19) may be classified as “semi-closed” because, although it forms a complete loop of material, the loop is breakable at either one of the sides or bottom end of the loop such that the medical tube that is held therein may easily be pulled from the restraint. The breakable portion of the loop may be provided by cutting the loop at any location. Such a cut may be provided at any number of angles relative to the loop.

A semi-closed cross-sectional guide configuration provides an improved gripping action for retaining the medical tube as compared to the open guide configuration. Also, the semi-closed bottom or side also allows for easier removal of the intubation device from the medical tube as compared to the closed configuration. In one embodiment, the distal tube restraint (18) comprises a semi-closed cross-sectional guide configuration. In another embodiment, the proximal tube restraint (14) comprises a semi-closed guide configuration. It is contemplated that one, more, or all distal tube restraints may comprise the semi-closed guide configuration. Furthermore, it is also contemplated that one, more, or all of the proximal tube restraints may also comprise the semi-closed guide configuration.

Each individual tube restraint, whether proximal or distal, may be independently selected to comprise the open guide configuration, the closed guide configuration, or the semi-closed guide configuration. In an embodiment, at least one distal tube restraint comprises an open guide or a semi-closed guide. In another embodiment, at least one proximal tube restraint comprises a closed guide or a semi-closed guide.

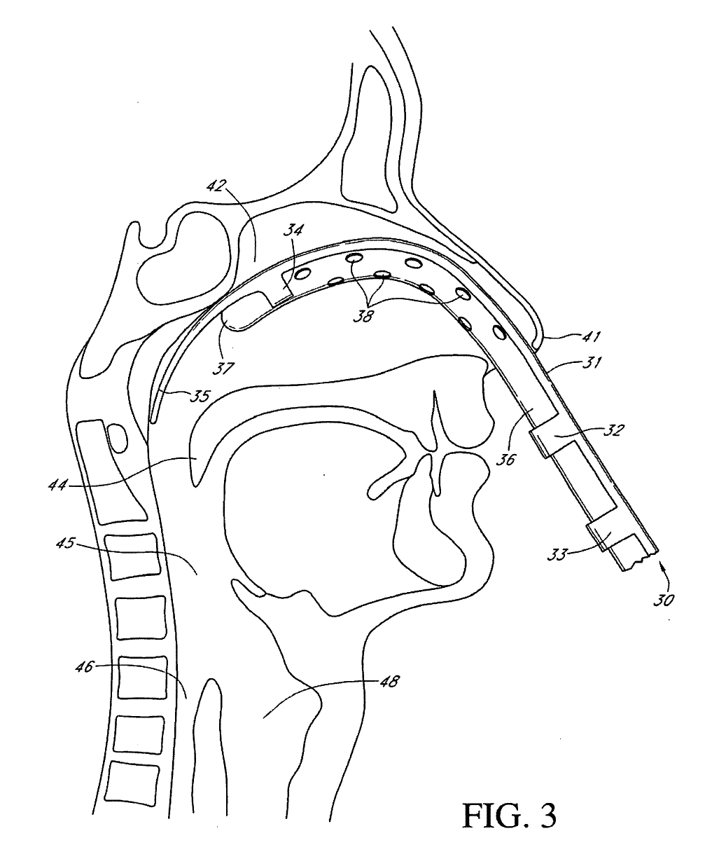

FIG. 2 shows the side view of an intubation device according to a non-limiting embodiment of the present invention. The intubation device (20) comprises a frame (22) comprising a distal region and a proximal region. A leader guide (26) is positioned at the distal region of the frame (22) and is in the form of a soft pliable curvature. The intubation device also comprises restraints (24, 25, 28) adapted to hold a medical tube.

FIG. 2 illustrates a preferred shape of the leader guide (26) of the intubation device (20). The leader guide (26) is in the form of a soft pliable curvature so that it may deflect off of a body cavity that it contacts. For example, when the medical tube is a nasogastric tube to be inserted into a patient's nose, the leader guide (26) is flexible enough to deflect off of the upper nasal cavity and is soft enough to not abrade the soft tissue of the upper nasal cavity. In one embodiment, the leader guide retains a curvature memory. This curvature memory may be formed when the intubation device (20) is created in a molding process or afterwards by heating the material and forming it into the curved shape until it hardens and maintains the shape permanently thereafter.

In an embodiment, the curvature of the leader guide (26) is shaped to permit the leader guide to follow the natural arch of a body cavity. The leader guide may be configured to substantially follow any mammalian body cavity where a medical tube is inserted. For example, in one non-limiting example, the leader guide may be formed so that it follows the natural arch of the upper nasal cavity.

The leader guide serves as a buffer between the medical tube and the body cavity to which the medical tube is being inserted. The leader guide gently contacts the tissue of the body cavity and the softness and curvature of the leader guide prevents damage and abrasion that is otherwise caused by a medical tube when the medical tube is inserted alone. Additionally, the leader guide also guides the medical tube into the proper openings and thus, lessens the chances that the medical tube is erroneously inserted into an area that results in damage to the patient.

FIG. 3 shows the cross-section of a human head when the intubation device is used to insert a medical tube. The intubation device (30) comprises a frame (31) comprising a distal region and a proximal region. The frame comprises proximal restraints (32, 33) and a distal restraint (34) adapted to hold a medical tube. The leader guide (35) is positioned at the distal region of the frame (31) and is in the form of a soft pliable curvature. Although FIG. 3 only shows the use of one distal restraint and two proximal restraints, other numbers of proximal and distal restraints are contemplated, as discussed above.

The intubation device may be removably connected or removably attached to a medical tube (36). In the embodiment illustrated in FIG. 3, the medical tube comprises a nasogastric tube. The medical tube comprises a distal region and a proximate region. The distal region of the medical tube comprises a distal end (37). In one embodiment, the distal region of the medical tube comprises drain holes (38) that are staggered in a spiral configuration.

The medical tube (36) and the intubation device (30) may be removably connected to one another for storage, shipping, marketing, or the like. The tube and intubation device may also be conveniently removably connected when packaged, so that the medical tube is pre-attached to the intubation device when removed from the package. Alternatively, the intubation device may be packaged separately and the medical tube may be removably attached thereto prior to insertion into the body.

Described herein is a nasogastric tube assembly comprising a intubation device that comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube; and a medical tube, preferably a nasogastric tube, comprising a distal region and a proximal region. The medical tube may be substantially straight when in a free-standing position, and is preferably substantially free of any curves, coils, or kinks. The medical tube size may range from about 8 Fr to about 24 Fr, and comprises a material selected from the group consisting of polyvinyl chloride, silicone, polyurethane, polyethylene, polypropylene, polycarbonate, polyester, polyacrylate, polytetrafluroroethylene, and combinations and copolymers thereof. In some embodiments, the medical tube is of a single lumen, and is substantially open, but it may also be substantially non-tapered, and the terminal end of the distal region of the tube may have an outer edge that has a rounded bead. In one embodiment, the distal region of the tube comprises a metal sleeve; in another, the distal region of the tube comprises one or more drain holes. If there is a plurality of drain holes, they are preferably staggered in a spiral configuration along the distal region of the tube, and are spaced at about 90 degree intervals along the spiral configuration.

In another embodiment, the nasogastric tube assembly comprises a intubation device that comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube; and a nasogastric tube, comprising a distal region and a proximal region, and the proximal region of the tube comprises a protecting shield. The protecting shield is, preferably, adapted to be connected to a patient's nose at a terminal end of the proximal region of the tube and may comprise an adhesive or an alternative means for adhering the protective shield to a patient's nose.

In another embodiment, the nasogastric tube assembly comprises an intubation device that comprises a longitudinal, flexible frame comprising a distal region and a proximal region, a leader guide positioned at the distal region of the frame, wherein said leader guide is in the form of a soft pliable curvature; and at least one restraint attached to the frame and adapted to hold a medical tube; and a nasogastric tube, comprising a distal region and a proximal region, and the nasogastric tube assembly tube further comprises a system for alerting when a blockage of the tube occurs.

The exemplary, depicted medical tube (36) in FIG. 3 is held to the intubation device using the proximal restraints (32, 33) and the distal restraint (34), all of which may have a closed, open, or semi-closed configuration. The distal end (37) of the medical tube fits near the area where the leader guide (35) of the intubation device begins to form a curvature. In one embodiment, the medical tube (36) will not contain any curvature that is caused by the leader guide (35).

The intubation device (30), having a medical tube (36) attached thereto, may be inserted into a body cavity, such as the upper nasal cavity, through a nostril of the patient's nose (41). As the intubation device enters the nostril, the leader guide (35) gently runs along the upper nasal cavity allowing the medical tube (36) to be guided into the nasal cavity without damaging contact of the medical tube (36) to soft tissue. The soft, pliable curvature of the leader guide (35) permits it to follow the natural arch of the upper nasal cavity preventing soft tissue abrasion.

The ethmoid bone is a bone in the skull that separates the nasal cavity from the brain and is located at the roof of the nose. The ethmoid region is a delicate region that can be prone to damage by improper insertion of a nasogastric tube. The leader guide (35) of the intubation device (30) is designed to follow the natural arch of the upper nasal cavity, making passage of the medical tube by the ethmoid region (42) much easier. The leader guide (35) greatly decreases the chances of the distal end (37) of the medical tube (36) penetrating the ethmoid region (42), resulting in less pain and abrasion, and further resulting in a less chance of severe brain damage or death.

As the administrator of the intubation device (30) pushes the device further into a patient's nasal cavity, the leader guide (35) is designed to continue to guide the intubation device (30) along the natural arch of the tissues therein. Thus, the medical tube (36) may be pushed past the soft palate (44) region that separates the nasal cavity from the mouth. The intubation device (30) is sufficiently flexible to conform to the shape of the body cavity to which it is being applied. The intubation device (30), however, is also sufficiently rigid to align and remain reasonably attached to the medical tube (36) throughout the process of insertion.

After the distal end (37) of the medical tube (36) reaches an area (45) that is midway between the soft palate (44) and the esophagus opening (46), the intubation device (30) may then be removed from the body by holding the medical tube (36) in place and gently withdrawing the intubation device (30). The intubation device (30) is then removed from the body. Once removed, the intubation device may be disconnected from the medical tube (36). Alternatively, the intubation device may be disconnected from the medical tube when both remain in the body. Where the proximal and distal restraints (32, 33, 34) of the intubation device (30) comprise open or semi-closed guide configurations, then the administrator may pull the intubation device (30) from the medical tube (36) using appropriate pressure. Where any of the restraints have a closed guide configurations, then the intubation device (30) may be removed from the medical tube (36) by cutting the restraints using an appropriate medical cutting device or other equipment capable of cutting polymer or plastic, for example, a pair of scissors.

After the intubation device (30) has been removed, the medical tube (36) may have its distal end (37) roughly in the area (45) of the midpoint between the soft palate (44) and the esophagus (46). From this point, the medical tube (36) is properly aligned for insertion into the esophagus (46). Thus, the chances of the medical tube (36) penetrating into the trachea (48) and causing damage to that area are significantly lessened.

In an alternative embodiment, the intubation device is designed to indicate to the administrator of the medical tube the point at which the distal end of the nasogastric tube has reached the area between the soft palate and the esophagus. In an embodiment that may be represented by FIG. 3, the intubation device (30) comprises a first proximal tube restraint (32) that is closer to a terminal end of the distal region of the intubation device than any other proximal tube restraint (33). The distance between the terminal end of the distal region of the intubation device and the first proximal tube restraint of the intubation device may be spaced apart so that when the intubation device is inserted into a nasal cavity, the terminal end of the distal region will be positioned between the soft palate region and the opening of the esophagus when the first proximal restraint is positioned at the opening of the nostril. That distance may vary, depending on the patient. In an embodiment, the distance between the terminal end of the distal region and the first proximal tube restraint is between about 4 cm and about 12 cm.

In another embodiment, the distal end of the medical tube is located in the region between the soft palate and the opening of the esophagus when the first proximal restraint is positioned at the opening of a patient's nostril. In such an embodiment, the intubation device will alert the administrator of the medical tube as to the proper moment to release the intubation device from the medical tube. Other embodiments of such an alert system are contemplated. For example, the intubation device may comprise a line, marking, or other indicia that will line up with the patient's nostril opening at the point where the distal end of the medical tube is in the region between the soft palate and the esophagus opening. Such a line, marking, or indicia may indicate the point where the intubation device may be removed from the medical tube so that the medical tube may be inserted on its own.

In another embodiment, the intubation device is a unitary structure made from a single piece of molded material such as plastic. The intubation device may be created using any number of molding techniques, including injection molding, compression molding, transfer molding, extrusion molding, blow molding, and rotational molding as non-limiting examples. One of ordinary skill in the art, guided by the disclosure provided herein, can fashion the proper mold and molding technique to create the intubation device described herein.

The intubation device described herein need not be made of a single unitary structure. For example, the intubation device may be formed of several distinct polymeric, plastic, or rubber pieces. These distinct pieces may be joined together using any known suitable bonding technique. The bonding technique should be medically suitable and may be, for instance, adhesive bonding or welding of the distinct pieces.

In an embodiment, the intubation device described herein further comprises a lubricant. The use of lubricants in inserting medical tubes into a patient is well known. For example, see U.S. Pat. No. 4,705,709 to Vailancourt, which is hereby incorporated by reference in its entirety. The lubricant can be used for providing easier attachment of the medical tube to the intubation device. Furthermore, lubrication may be used on both the medical tube and the intubation device to help guide the combination into a patient's body cavity.

In another embodiment, the present invention describes a nasogastric tube assembly comprising a combination of an intubation device described herein and a medical tube that comprises a nasogastric tube having a distal region and a proximal region. The intubation device described herein allows for various improvements to medical tubes and nasogastric tubes, as described herein.

Medical tubes, including nasogastric tubes, often exhibit a curvature memory. The curvature memory may be induced during manufacture of the tube as the molten plastic that comprises the tube hardens while being placed on a coil. Memory curvature has often been applied to nasogastric tubes because it has been thought that such curvature facilitates the ability to negotiate the tube through the nasal cavity. However, nasogastric tubes with a memory curve still are likely to abrade the soft tissues. Additionally, the chance of the tube penetrating the ethmoid region and moving into the brain still exists. Furthermore, the curvature in the nasogastric tube magnifies the difficulty of locating the tube in the esophagus, rather than the trachea.

In another embodiment, the medical tube described herein is substantially straight when in a free-standing position. As used herein, “substantially straight” means that the medical tube, which is usually some form of plastic, maintains a substantially parallel alignment with itself unless a force is applied to it which makes it form some type of curve or kink. Preferably, the tube is substantially free of any curves, coils, or kinks. As shown in FIG. 3, after the intubation device guides the nasogastric tube into the region (45) between the soft palate (44) and the esophagus (46) and is removed, a substantially straight nasogastric tube will allow for a direct line up of the distal end (37) of the medical tube (36) with the esophagus (46). This avoids the common problem of nasogastric tubes entering the trachea (48) and causing damage to the tissues therein and also the lungs.

The intubation device described herein is adaptable to be used with almost any type of inserted medical tube. The intubation device described herein works equally well with those nasogastric tubes that do possess some amount of curvature. In one embodiment, the medical tube has curvature.

The medical tube described herein may comprise various dimensions. In an embodiment, the tube size ranges from about 8 Fr to about 24 Fr. In another embodiment, the tube size ranges from about 12 Fr to about 18 Fr. The radius of the tube may vary depending on the patient and the procedure required. For example, a smaller radius tube, such as 12 Fr or any other suitable size, may be used with a child patient, whereas a larger radius tube, such as 18 Fr, may be used with an adult patient. The intubation device may also be useful for even larger tubes, such as 30 Fr, 36 Fr, or larger.

The medical tube may be made from any material that is commonly used in the medical tube or nasogastric tube field. Some non-limiting examples of materials useful in manufacturing the tube include polyvinyl chloride, silicone, polyurethane, polyethylene, polypropylene, polycarbonate, polyester, polyacrylate, ethylene vinyl acetate copolymer, polyvinyl pyrol, polytetrafluroroethylene, and combinations and copolymers thereof. The manufacture of medical tubes is well known in the art. For example, see the disclosures of U.S. Pat. No. 6,193,699 to Matsumoto et al.; U.S. Pat. No. 5,700,252 to Klingenstein; U.S. Pat. No. 5,690,620 to Knott; U.S. Pat. No. 4,692,152 to Emde; and U.S. Pat. No. 3,971,385 to Corbett; all of which are hereby incorporated by reference in their entirety.

In one embodiment, the medical tube comprises a single lumen. In another embodiment, the medical tube comprises multiple lumens. For example, the medical tube may comprise two, three, four, or more lumens.

The intubation device described herein allows medical tubes, particularly nasogastric tubes, to comprise various feature not previously thought to be available. For example, nasogastric tubes commonly have coned and closed off distal end so as to minimize tissue abrasion in the nasal cavity. However, the intubation device described herein guides the medical tube past the nasal cavity whether the medical tube has an open or a closed and tapered distal end. In an embodiment, the terminal end of the distal region of the medical tube is substantially open. In another embodiment, the terminal end of the distal region of the medical tube is completely open. Providing an opening at the terminal end of the distal region of the medical tube allows the tube to have increased suction capabilities when the tube is placed within a patient's stomach. In another embodiment, the terminal end of the distal region of the tube is substantially non-tapered.

Where the medical tube has a terminal end in the distal region that is open, the edges of the open end may be rounded so as to minimize abrasion resulting from incidental contact with tissue. In an embodiment, the terminal end of the distal region of the tube has an outer edge that has a rounded bead.

When a nasogastric tube has been positioned in the patient's stomach or upper small intestine, a monitoring system may be used to determine if the distal end of the tube is in a suitable location. In an embodiment, the distal region of the medical tube comprises a metal sleeve. A metal sleeve may be in the form of a small ring of metal that encircles the medical tube. Any biocompatible metal may be used. The metal sleeve allows a technician to easily monitor where the end of the tube is located using any type of monitoring technique that is capable of detecting metal.

In order to increase suction of the contents within the stomach, the medical tube may be provided with drain holes (38), as illustrated in FIG. 3. In an embodiment, the drain holes are staggered in a spiral configuration. Such a configuration ensures that when part of the medical tube is pressed against the wall of the stomach, at least some portion of the medical tube will still be exposed to liquids and will enable suction. In another embodiment, the drain holes are spaced at anywhere between 45 and 180 degree intervals along the spiral configuration. In another embodiment, the drain holes are spaced at 90 degree intervals along the spiral configuration. Staggering the drain holes at 90 degree intervals along the spiral configuration allows for drainage from four different directions, while maintaining sufficient tube wall strength to prevent buckling of the tube during drainage.

Once the distal end of the nasogastric tube is properly located in a patient's stomach, the proximal end may be attached to a suction manifold that enables collection of the stomach contents. Nasogastric aspiration is the process of draining the contents of the stomach via the nasogastric tube. Aspiration may be used to remove gastric secretions, air, poisoning, preparation for surgery under anesthesia, and to extract samples of gastric liquid for analysis. The tube may be appended to a collector bag placed below the level of the patient's stomach to allow gravity to empty the contents. Alternatively, the nasogastric tube may be applied to a suction system.

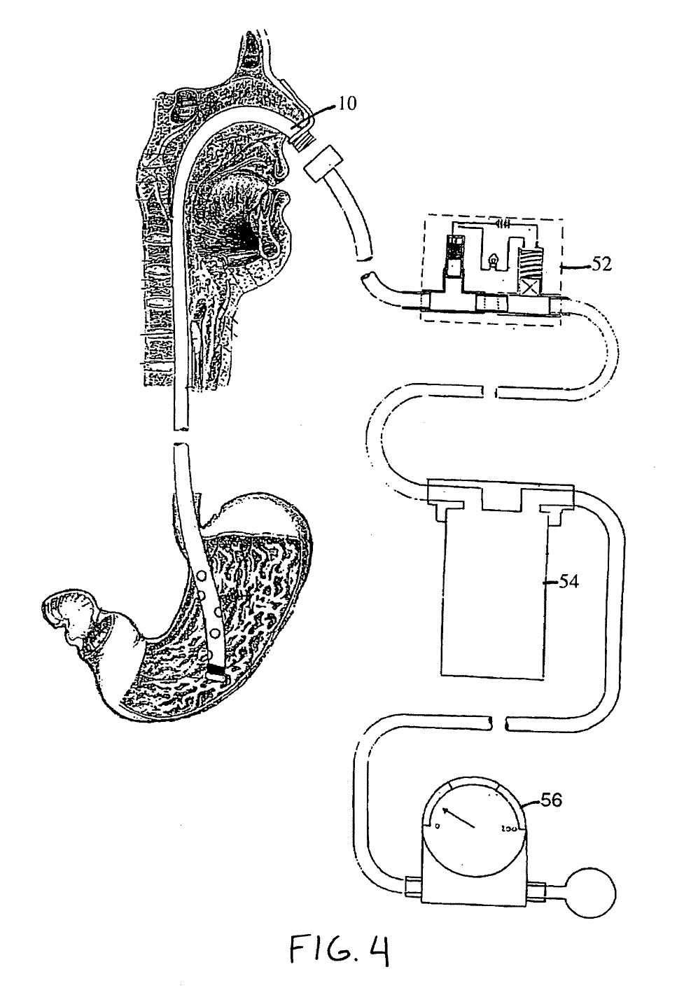

The nasogastric tube may be used for feeding, administrating drugs, and other various agents. A gravity based system may be employed by placing the contents to be administered higher than the stomach of the patient. Additionally, the tube may be connected to a pump which can administer the contents accordingly. FIG. 4 illustrates the components of an embodiment utilizing a blockage detector and shut off (52), a collection canister (54), a vacuum regulator (56) and the intubation device (30) shown placed within a patient. This embodiment is shown using an intubation device (30) having a substantially straight tube with spiraled inlet ports, a blunted and open distal end, a metal detected position ring, and a nose disconnect and retainer. Also shown in this embodiment is a blockage detector and shut off (52) having a warning light to indicate the presence or possible presence of a blockage in the device (30), a system shutoff for manual and/or automatic shutoff of the device, a manual reset switch, button or other similar signal for the device (30) and an applied pressure regulator. In this embodiment, a negative reflux canister is also provided.

The location of the terminal end of the proximal region of the medical tube may vary. Typically, the medical tube may extend outside the patient's body to a midway point between the patient and the device to which the medical tube is connected. The problem with such an arrangement includes the likelihood that a patient may pull on the exterior of the tube and thus interfere with its interior positioning within the body. Additionally, an ambulatory patient must move around with the embarrassment of having an extended length of tube protruding from the nose. Also, the extended length of tube makes it more difficult to clear a blockage within the tube. Such blockages not only inhibit the suction capabilities, but they also inhibit administration of food or drugs when the tube is used as an administration device rather than a removal device.

In order to guard against such disadvantages, it is preferred that the terminal end of the proximal region be provided at the nasal opening. In order to achieve this proposed embodiment, the proximal region of the medical tube comprises a protecting shield. The protecting shield is adapted to be connected to a patient's nose at a terminal end of the proximal region of the tube. Thus, when the medical tube comprises a nasogastric tube, the tube will have very little or no length that is external to the human body. Such an embodiment lessens the chance that the tube will be misplaced within the body by an inadvertent or intentional force exerted on the external area of the tube. Additionally, the protecting shield can be masked for increased patient comfort and appearance.

In an embodiment, the protecting shield comprises a port for connecting and disconnecting the external tubes to the internal medical tube. Such a port may take the form of known devices and materials that connects medical tubes together.

In an embodiment, the protective shield comprises a means for adhering the protective shield to a patient's nose. The protective shield may adhere to the nose using an adhesive. In an embodiment, the adhesive comprises any adhesive that is compatible with skin and provides secure attachment. In another embodiment, the protective shield may adhere to the nose using a clamp, a pin, adhesive tape, sutures, threading, or combinations thereof.

In an embodiment, the location of the protective shield on the length of medical tubing will be adjustable. Where the distal end of the medical tube reaches the targeted end location within the body such as, for instance, the stomach or upper intestinal tract, the protective shield can move along the medical tubing to the opening of a patient's nose. In such an instance, an activatable adhesive may be used. An activatable adhesive is any adhesive that takes on its adhesive characteristics upon application or mixture with another substance such as, for instance, water. The proximal end of the tube may then be cut so that the proximal region of the tube ends in a location at or near the patient's nose. The protective shield may then attach the terminal end of the proximal region of the tube to the nose.

In another embodiment, a method for accurately detecting tube blockage is provided. If the nasogastric tube is properly assembled to a suction manifold and collection canister, the entire assembly may further comprises a system for alerting when a blockage of the tube occurs. In one embodiment, a visual display unit may be connected to the suction manifold that activates upon sensing a change in air pressure during suction. Such a system enables more efficient use of nasogastric tubes.

In an embodiment, a medical tube is adapted to be used with an intubation device that comprises a longitudinal, flexible frame comprising a distal region and a proximal region; a leader guide positioned in the distal region of the frame, wherein said leader guide is in the form of a soft pliable curvature; and at least one restraint attached to the frame and adapted to hold a medical tube. In one embodiment, the medical tube comprises a nasogastric tube.

Another embodiment includes a medical kit comprising packaging material an intubation device comprising a longitudinal, flexible frame comprising a distal region and a proximal region; a leader guide positioned in the distal region of the frame, wherein said leader guide is in the form of a soft pliable curvature; and at least one restraint attached to the frame and adapted to hold a medical tube; and a medical tube. The packaging material may be that which is typically used to package medical devices. Preferably, the packaging material provides a sterile environment for the intubation device and the medical tube. In one embodiment, the medical tube in the medical kit comprises a nasogastric tube.

Another embodiment is a method of inserting a medical tube comprising providing an intubation device comprising a longitudinal, flexible frame comprising a distal region and a proximal region; a leader guide positioned in the distal region of the frame, wherein said leader guide is in the form of a soft pliable curvature; and at least one restraint attached to the frame and adapted to hold a medical tube; providing a medical tube comprising a distal region and a proximal region; fitting the tube into the intubation device; inserting the intubation device and the tube into a nasal cavity such that the terminal end of the distal region of the tube is located between the soft palate region and the opening of the esophagus; and withdrawing the intubation device while holding the tube in place.

In one embodiment of the method of inserting a medical tube, the medical tube is a nasogastric tube. In another embodiment, the method further comprises the step of applying lubrication to areas of the distal region of the nasogastric tube. In another embodiment, the method further comprises the step of inserting the medical tube into the stomach through the esophagus. In still a further embodiment, the method of inserting the medical tube will not include the step of fitting the medical tube into the intubation device because the combination of the tube and the intubation device will already be packaged together and ready to use.

All patents incorporated by reference herein are incorporated by reference herein only with respect to the particular embodiments, materials, processes of manufacture and methods of use described therein. These patent are not to be considered incorporated by reference to the extent any of these patents expresses an opinion or presents any representation, characterization, or definition (either expressly or by implication) that is inconsistent with the opinions, representations, characterizations or definitions expressly made herein.

While there have been described herein what are to be considered exemplary and preferred embodiments of the present invention, other modifications of the invention will become apparent to those skilled in the art from the teachings herein. For example, the leader guide may take on various shapes to allow it to conform to the shape of a body cavity. The curvature of the leader guide may be appropriately adjusted to guide a medical tube through body cavities and organs that range from substantially straight to highly curved in their nature. For instance, where the intubation device is to guide a medical tube into a body cavity comprising highly angled curves, the leader guide may be provided with a greater amount of curvature.

The number of distal and proximal restraints may be increased to any number. For instance, providing more than ten distal tube restraints and more than ten proximal tube restraints is contemplated where the size dimensions of several of the restraints is substantially small.

The stiffness of the intubation device may be adjusted according to the relative stiffness of the medical tubing to be inserted. The medical device should be stiff enough to align and removably hold a medical tube in place while maintaining the ability to curve the medical tube around a body cavity. Various portions of the intubation device may comprise a stiffness that is different from another portion of the intubation device. For instance, the proximal tube restraints may have a stiffness that is greater than the stiffness of the distal tube restraints. Additionally, the leader guide may have a stiffness that is greater than the stiffness of the frame.

The medical tube may comprise many other configurations. For example, the metal to detect the location of the medical tube may be in the form of longitudinally disposed slivers of metal and may be imbedded into the tube. Alternatively, the medical tube or distal region thereof may further include a radiopaque stripe, band, or other marker to facilitate fluoroscopic verification of the location of the tube in the body of a patient.

The drain holes of the medical tube may comprise any configuration allowing for sufficient suction or administration of gases, liquids, and solids. For instance, the drain holes may be shaped substantially circular, substantially elliptical, substantially triangular, substantially rectangular, or any other polygonal shape and combinations thereof. The medical tube may comprise any number of lumens, include up to five, six, seven, eight, nine, or even ten lumens. Preferably, the medical tube will be of sufficient stiffness to prevent buckling after it is inserted into the body and used in its appropriate function.

It is therefore desired to be secured in the appended claims all such modifications as fall within the true spirit and scope of the invention. Accordingly, what is desired to be secured by Letters Patent is the invention as defined and differentiated in the following claims.

What is claimed is:

1. An intubation device comprising:
a longitudinal, flexible frame comprising a distal region and a proximal region;
a leader guide positioned at the distal region of the frame, wherein said leader guide is in the form of a soft pliable curvature; and
at least one restraint attached to the frame and adapted to hold a medical tube.

2. The intubation device according to claim 1, wherein the intubation device comprises a plastic or rubber material.

3. The intubation device according to claim 2, wherein the plastic material is selected from the group consisting of polyvinyl chloride, silicone, polyurethane, polyethylene, polypropylene, polycarbonate, polyester, polyacrylate, polytetrafluroroethylene, and combinations and copolymers thereof.

4. The intubation device according to claim 1, wherein the frame maintains a substantial lineal integrity.

5. The intubation device according to claim 1, wherein the frame is capable of a 180 degree direction reverse within about a 2 cm radius or less.

6. The intubation device according to claim 1, wherein the frame is capable of a 180 degree direction reverse within about a 1 cm radius or less.

7. The intubation device according to claim 1, wherein the leader guide retains a curvature memory.

8. The intubation device according to claim 1, wherein the curvature of the leader guide is shaped to permit the leader guide to substantially follow the natural arch of a nasal cavity.

9. The intubation device according to claim 1, wherein one or more of the at least one restraint comprises a substantially circular or substantially elliptical shape.

10. The intubation device according to claim 1, wherein one or more of the at least one tube restraint is independently selected from the group comprising a substantially closed guide, a substantially semi-closed guide, and a substantially open guide.

11. The intubation device according to claim 1, wherein the intubation device comprises at least one distal tube restraint positioned in the distal region of the frame and at least one proximal tube restraint positioned in the proximal region of the frame.

12. The intubation device according to claim 11, wherein the at least one distal tube restraint and the at least one proximal tube restraint are adapted to maintain substantial lateral alignment of a medical tube with the intubation device.

13. The intubation device according to claim 11, wherein one or more of the at least one distal tube restraint comprises a substantially open guide or a substantially semi-closed guide.

14. The intubation device according to claim 12, wherein the at least one proximal tube restraint comprises a substantially closed guide or a substantially semi-closed guide.

15. The intubation device according to claim 13, wherein the at least one proximal tube restraint comprises a substantially closed guide or a substantially semi-closed guide.

16. The intubation device according to claim 11, wherein the intubation device comprises one, two, or three distal tube restraints.

17. The intubation device according to claim 11, wherein the intubation device comprises one, two, or three proximal tube restraints.

18. The intubation device according to claim 11, wherein the intubation device comprises a first proximal tube restraint that is closer to a terminal end of the distal region of the intubation device than any other proximal tube restraint.

19. The intubation device according to claim 11, wherein the distance between the terminal end of the distal region and the first proximal tube restraint is adapted so that when the intubation device is inserted into a nasal cavity, the terminal end of the distal region will be positioned between the soft palate region and the opening of the esophagus when the first proximal restraint is positioned at the opening of the nostril.

20. The intubation device according to claim 1, wherein the intubation device is a unitary structure.

21. The intubation device according to claim 1, further comprising a lubricant.

22. The intubation device according to claim 1, wherein the intubation device is adapted to hold a nasogastric tube.

23. The intubation device according to claim 1, further comprising a medical tube comprising a distal region and a proximal region.

24. The intubation device according to claim 23, wherein the intubation device is a nasogastric tube.

25. The intubation device according to claim 23, wherein the tube is substantially straight when in a free-standing position.

26. The intubation device according to claim 23, wherein the tube is substantially free of any curves, coils, or kinks.

27. The intubation device according to claim 23, wherein the tube size ranges from about 8 Fr to about 24 Fr.

28. The intubation device according to claim 23, wherein the tube is made from a material selected from the group consisting of polyvinyl chloride, silicone, polyurethane, polyethylene, polypropylene, polycarbonate, polyester, polyacrylate, polytetrafluroroethylene, and combinations and copolymers thereof.

29. The intubation device according to claim 23, wherein the tube is comprised of a single lumen.

30. The intubation device according to claim 23, wherein a terminal end of the distal region of the tube is substantially open.

31. The intubation device according to claim 23, wherein a terminal end of the distal region of the tube is substantially non-tapered.

32. The intubation device according to claim 23, wherein a terminal end of the distal region of the tube has an outer edge that has a rounded bead.

33. The intubation device according to claim 23, wherein the distal region of the tube comprises a metal sleeve.

34. The intubation device according to claim 23, wherein the distal region of the tube comprises at least one drain hole.

35. The intubation device according to claim 34, wherein the at least one drain hole is a plurality of drain holes and wherein the drain holes are staggered in a spiral configuration.

36. The intubation device according to claim 35, wherein the holes are spaced at 90 degree intervals along the spiral configuration.

37. The intubation device according to claim 23, wherein the proximal region of the tube comprises a protecting shield.

38. The intubation device according to claim 37, wherein the protecting shield is adapted to be connected to a patient's nose at a terminal end of the proximal region of the tube.

39. The intubation device according to claim 37, wherein the protecting shield comprises an adhesive.

40. The intubation device according to claim 37, wherein the protective shield comprises a means for adhering the protective shield to a patient's nose.

41. The intubation device according to claim 37, wherein the intubation assembly tube further comprises a system for alerting when a blockage of the tube occurs.

42. A medical tube that is adapted to be used with an intubation device that comprises a longitudinal, flexible frame comprising:
a distal region and a proximal region;
a leader guide positioned in the distal region of the frame, wherein said leader guide is in the form of a soft pliable curvature; and
at least one restraint attached to the frame and adapted to hold a medical tube.

43. The medical tube according to claim 0, wherein said medical tube comprises a nasogastric tube.

44. The medical tube according to claim 0, wherein the flexible frame, leader guide, and at least one restraint form a unitary device.

45. A medical kit comprising:
packaging material;
an intubation device comprising a longitudinal, flexible frame comprising a distal region and a proximal region; a leader guide positioned in the distal region of the frame, wherein said leader guide is in the form of a soft pliable curvature; and at least one restraint attached to the frame and adapted to hold a medical tube; and
a medical tube.

46. The medical kit according to claim 0, wherein the medical tube comprises a nasogastric tube.

47. A method of inserting a medical tube comprising:
providing an intubation device comprising a longitudinal, flexible frame comprising a distal region and a proximal region; a leader guide positioned in the distal region of the frame, wherein said leader guide is in the form of a soft pliable curvature; and at least one restraint attached to the frame and adapted to hold a medical tube;
providing a medical tube comprising a distal region and a proximal region;
fitting the tube into the intubation device;
inserting the intubation device and the tube into a nasal cavity such that the terminal end of the distal region of the tube is located between the soft palate region and the opening of the esophagus; and
withdrawing the intubation device while holding the tube in place.

48. The method according to claim 0, wherein the medical tube is a nasogastric tube.

49. The method according to claim 0, further comprising the step of applying lubrication to areas of the distal region of the medical tube.

50. The method according to claim 0, further comprising the step of inserting the medical tube into the stomach through the esophagus.
